(19) United States
(12) Patent Application Publication
(10) Pub. No.: US 2022/0165436 A1
(43) Pub. Date: May 26, 2022

(54) Method and System for Optimizing Research and Development Experimentations

(71) Applicant: AIZEN ALGO PRIVATE LIMITED, Hyderabad, Telangana (IN)

(72) Inventors: Usha Balakrishnan Bithunshal, Hyderabad, Telangana (IN); Varma Srinivasa Rajasekara Rudraraju, Hyderabad, Telangana (IN); Ramdas Manakkote, Hyderabad, Telangana (IN)

(21) Appl. No.: 17/440,962

(22) PCT Filed: Mar. 21, 2020
(86) PCT No.: PCT/IN2020/050267

§ 371 Date: Sep. 20, 2021

(30) Foreign Application Priority Data
Mar. 21, 2019 (IN) 201841035671

(51) Int. Cl. G16H 70/40; G16H 50/20; G16H 40/20

(52) U.S. Cl. CPC G16H 70/40; G16H 40/20; G16H 50/20

(57) ABSTRACT

The present disclosure relates to a method and system for optimizing research and development experimentations. The method comprises receiving characteristics of reference listed drug (RLD) and Active Pharmaceutical Ingredient (API) associated with the RLD and identifying a manufacturing process for the pharmaceutical product based on the API and characteristics of RLD received. The method further comprises generating notifications to at least one or more users to develop the pharmaceutical product using the API, one or more excipients associated with the API, and the identified manufacturing process. The method also comprises determining acceptance range of one or more values associated with properties of the pharmaceutical product and optimizing research and development experimentations based on the determination.

### CROSS REFERENCE TO RELATED APPLICATION

This application claims the benefit of priority to Indian Provisional Patent Application Number 201841035671, filed on Mar. 21, 2019, the entire contents of which are hereby incorporated by reference.

### TECHNICAL FIELD

Embodiments of the present disclosure are related, in general to resource management systems, and more particularly, but not exclusively to a method and system for optimizing research and development experimentations.

### BACKGROUND

Generally, research & development (R&D) operations in an organization involve manual operations carried out on unconnected technology platforms. In R&D operations, number of experimental iterations while conducting research is not under a control of researcher as the observations or inferences from the experimental iterations will only decide the next steps or experimental processes. Also, among factors that seriously affect a process, only few factors would be critical depending on specific product being developed. Currently, identifying the critical factors in the R&D practice bring unwanted economic burden to industries that often result in delay of time bound functioning of the industry. For example, in manufacturing a drug product, selection of proper manufacturing method is a critical stage of the product development which will eventually decide the success of the product development. Currently, selection of the manufacturing method is purely based on the experience and knowledge of the researcher involved or based on knowledge in publicly available literature. Only upon conducting several experiments or upon monitoring stability of the product for a few months, the researcher or developer would be able to predict the suitability of the experiments or select suitable manufacturing method which is one of major challenges in a pharmaceutical drug development. Also, in an R&D environment, different researchers adopt different experimental approaches while developing the product, which leads to diverse working or experimentation culture across organization. In order to avoid diversities in development across an organization, an intelligent system is needed to help the researcher to deliver products, for example pharmaceutical products in an efficient way. Currently, there is no such system and techniques available that control end to end aspects of pharmaceutical product development with a limited number of R&D experiments and which assists in important science-based decision making in all phases of a product development.

Accordingly, there is required an intelligent technique to provide a method and system for optimizing research and development experimentations.

### SUMMARY

Embodiments of the present disclosure relate to a method of optimizing research and development experimentations. The method comprises receiving characteristics of reference listed drug (RLD) and Active Pharmaceutical Ingredient (API) associated with the RLD and identifying a manufacturing process for the pharmaceutical product based on the API and characteristics of RLD received. The method further comprises generating notifications to at least one or more users to develop the pharmaceutical product using the API, one or more excipients associated with the API, and the identified manufacturing process. The method also comprises determining acceptance range of one or more values associated with properties of the pharmaceutical product and optimizing research and development experimentations based on the determination.

Another aspect of the present disclosure relates to a system for optimizing research and development experimentations. The system comprises a memory and a processor. The processor is configured to receive characteristics of reference listed drug (RLD) and Active Pharmaceutical Ingredient (API) associated with the RLD and identify a manufacturing process for the pharmaceutical product based on the API and characteristics of RLD received. The processor is further configured to generate notifications to at least one or more users to develop the pharmaceutical product using the API, one or more excipients associated with the API, and the identified manufacturing process. The processor is also configured to determine acceptance range of one or more values associated with properties of the pharmaceutical product and optimize research and development experimentations based on the determination.

The system, and associated method of the present disclosure overcome one or more of the shortcomings of the prior art. Additional features and advantages may be realized through the techniques of the present disclosure. Other embodiments and aspects of the disclosure are described in detail herein and are considered a part of the claimed disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device or system and/or methods in accordance with embodiments of the present subject matter are now described, by way of example only, and with reference to the accompanying figures, in which:

FIG. 1 illustrates an exemplary architecture of a proposed system, to optimize research and development experimentation, in accordance with some embodiments of the present disclosure;

FIG. 2 illustrates various components and modules of resource optimization system (ROS), for pharmaceutical product development, in accordance with some embodiments of the present disclosure;

FIG. 3 illustrates an exemplary flow diagram for a pharmaceutical product development in accordance with some embodiments of the present disclosure; and

FIG. 4 illustrates a block diagram of an exemplary computer system for implementing embodiments consistent with the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

The present disclosure relates to a system and method of optimizing research and development experimentations. The method comprises a comprehensive workflow that enables implementation of minimum number of R&D experiments in all phases of the research and development. The reduction in the number of R&D experiments is achieved by using the experimental knowledge and historical experimental results for optimizing the steps of manufacturing process, thereby optimizing research and development experimentations.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

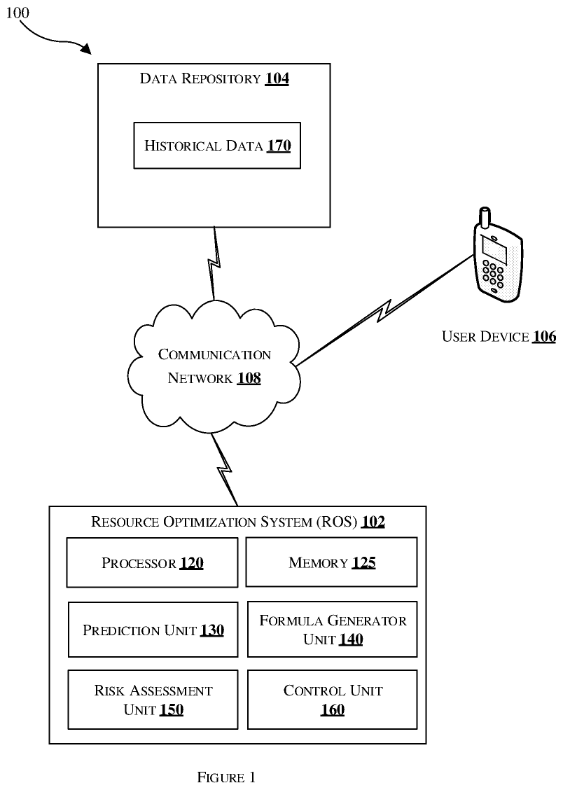

FIG. 1 illustrates an exemplary architecture of a proposed system, to optimize research and development experimentation, in accordance with some embodiments of the present disclosure.

As illustrated in FIG. 1, the exemplary system 100 comprises one or more components configured to optimize resources for a pharmaceutical product development. In one embodiment, the exemplary system 100 comprises a resource optimization system (ROS) 102, a data repository 104 (interchangeably referred to as repository 104), and a user device 106 coupled via a communication network 108.

The ROS 102 is configured to optimize resources for development of the pharmaceutical product by establishing an effective workflow for the R&D processes. In one example embodiment, optimizing resources include but not limited to minimizing the R&D experimentations. In one embodiment, the ROS 102 comprises one or more mathematical, statistical and scientific tools that aids the researcher or user to quickly make efficient scientific decisions on selection of resources. The ROS 102 comprises one or more components coupled with each other that may be deployed on a single system or on different systems. In an embodiment, the ROS 102 comprises a central processing unit (“CPU” or “processor”) 120, a memory 125, a prediction unit 130, a formula generator unit 140, a risk assessment unit 150, and a control unit 160.

The ROS 102 may be configured as a standalone system. In another embodiment, the ROS 102 may be configured in cloud environment. In yet another embodiment, the ROS 102 may include a desktop personal computer, workstation, laptop, PDA, cell phone, or any WAP-enabled device or any other computing device capable of interfacing directly or indirectly with the Internet or other network connection. The ROS 102 typically includes one or more user interface devices, such as a keyboard, a mouse, touch screen, pen or the like, for interacting with the Graphical User Interface (GUI) provided on a display. The ROS 102 also includes an interface provided therein for interacting with the repository 104 and the user device 106 to access data from the repository 104 for R&D process optimization.

The data repository 104 stores previously recorded data of one or more earlier experimentations as historical data 170. In one example, the historical data 170 may be an experimental data collected from a plurality of experiments previously conducted on the same or similar pharmaceutical product. The historical data 170 also stores acceptance criteria and regulatory restrictions for parameters of formulation, such as Inactive Ingredient Guide (IIG) specification. The IIG specification describes allowable limits of one or more excipients for a development of the pharmaceutical products. The ROS 102 is configured to access the data repository 104 to retrieve the historical data 170 and the IIG specification during the resource optimization process. In one embodiment, the data repository 104 may be integrated within the ROS 102. In another embodiment, the data repository 104 may be a standalone repository communicatively coupled with the ROS 102 and the user device 106. The user device 106 comprises an integrated application that enables interaction of user with the ROS 102 via a user-friendly application interface. For example, the integrated application of the user device 106 enables the user to input one or more values such API values, characteristic values of reference listed drug (RLD) to the ROS 102. The integrated application of the user device 106 wirelessly connects to the ROS 102, to receive alerts or notification from the ROS 102. In another embodiment, the user device 106 connects with the ROS 102 over Transfer Control Protocol and Internet Protocol (TCP/IP) via the communication network 108.

The communication network 108 can be a LAN (local area network), WAN (wide area network), wireless network, point-to-point network, or another configuration. One of the most common types of network in current use is a TCP/IP (Transfer Control Protocol and Internet Protocol) network for communication between database client and database server. Other common Internet protocols used for such communication include HTTPS, FTP, AFS, and WAP and using secure communication protocols etc.

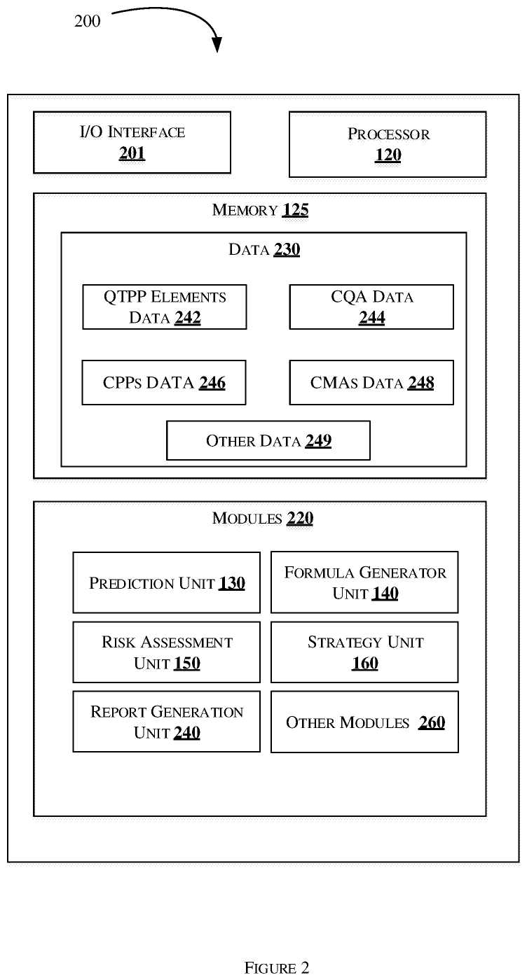

FIG. 2 illustrates various components and modules of ROS, for a pharmaceutical product development, in accordance with some embodiments of the present disclosure.

In an implementation, the ROS 102 may include an I/O interface 201, the processor 120, the memory 125, and modules 220. The I/O interface 201 may be configured to receive inputs from one or more users for optimizing resources for the pharmaceutical product development. Further, the I/O interface 201 may be configured to communicate with the repository 104 and the user device 106. The processor 120 may be configured to perform one or more functions of the ROS 102 for optimizing resources for the pharmaceutical product development. The memory 125 may be communicatively coupled to the processor 120 and may store data 230.

In an embodiment, the data 230 may include, without limiting to, QTPP elements data 242, CQA data 244, CPPs data 246, and CMAs data 248, and other data 249.

In one embodiment, the Quality Target Product Profile (QTPP) elements data 242 may be retrieved from the characteristics of reference listed drug (RLD) using the historical database 170. In an exemplary embodiment, the RLD is also known as innovator drug. The QTTP elements data 242 for example, includes dosage form, dosage design, route of administration, dosage strength, pharmacokinetics, stability, drug product quality attributes, container closure system, and alternative methods of administration. In one embodiment, the drug product quality attributes comprise physical attributes, identification, assay content uniformity, dissolution, degradation products, residual solvents, water content and microbial limits. In one embodiment, the physical attributes comprise appearance, order, size, score configuration and friability.

In one embodiment, the critical quality attribute (CQA) data 244 includes at least one of physical, chemical, biological, or microbiological property or characteristic of the pharmaceutical product that should be within an appropriate limit, range, or distribution to ensure a desired product quality of the pharmaceutical product.

In one embodiment, the critical process parameters (CPPs) data 246 includes one or more process parameters whose variability has an impact on the CQA data 244 and therefore should be monitored or controlled to ensure that the process produces the desired quality.

In one embodiment, the critical material attributes (CMAs) data 248 includes at least one of physical, chemical, biological, or microbiological property or characteristic of the pharmaceutical product whose variability has an impact on the CQA data 244 and therefore should be monitored or controlled to produce the desired quality.

In some embodiments, the data 230 may be stored within the memory 125 in the form of various data structures. Additionally, the data 230 may be organized using data models, such as relational or hierarchical data models. The other data 249 may comprises other temporary data generated by other modules 260 for performing various functions of the ROS 102.

In some embodiments, the ROS 102 may include the modules 220 for performing various operations in accordance with embodiments of the present disclosure. The modules 220 may include, for example, the prediction unit 130, the formula generator unit 140, the risk assessment unit 150, the strategy unit 160, and a report generation unit 240. The modules 220 may also comprise other modules 260 to perform various miscellaneous functionalities of the ROS 102. It will be appreciated that such modules 260 may be represented as a single module or a combination of different modules. The modules 220 may be implemented in the form of software, hardware, and/or firmware. As used herein, the term module refers to an application specific integrated circuit (ASIC), an electronic circuit, a processor (shared, dedicated, or group) and memory that execute one or more software or firmware programs, a combinational logic circuit, and/or other suitable components that provide the described functionality.

In an embodiment, the prediction unit 130 is configured to receive characteristics of RLDs and the Active Pharmaceutical Ingredient's (APIs) inputted by one or more users. Upon receiving the characteristics of RLDs, the prediction unit 130 identifies CQA data 244 and QTPP elements data 242 for the pharmaceutical product to be developed. The prediction unit 130 also predicts an appropriate manufacturing process, by verifying if one or more APIs meet predefined criteria for the pharmaceutical product based on the historical data 170 for the same or similar RLDs. In an example, the predefined criteria can be the weight percentage of the one or more APIs in the pharmaceutical product. The prediction unit 130 determines the manufacturing process as wet granulation if the weight percentage of the one or more APIs data in the pharmaceutical product exceeds 30%. Once the manufacturing process is determined, the prediction unit 130 determines one or more excipients data and composition of each of the one or more excipients to be used for developing the pharmaceutical product based on the historical data 170 for API's of the same or similar RLDs. The prediction unit 130 also generates notifications to the one or more users to develop the pharmaceutical product and determines acceptance range of one or more values associated with properties of the pharmaceutical product based on the historical data 170 for API's of the same or similar RLDs. The prediction unit 130 also receives the one or more values associated with properties of the pharmaceutical product to be developed from the one or more users. For example, the properties of the pharmaceutical product may be, but not limited to hardness, friability, weight, and sticking. The prediction unit 130 modifies the manufacturing process for the pharmaceutical product upon determination that the one or more values exceed the acceptance range. In an example, the predicted unit 130 modifies the manufacturing process for example, from the wet granulation process to a direct compression method upon determination that the one or more values exceeded the acceptance range.

The formula generator unit 140 receives the one or more excipients data associated with the API data from the prediction unit 130 and calculates weight per tablet for each of the one or more excipients data. The formula generator unit 140 further determines qualification of the one or more excipients to be used in the pharmaceutical product development by comparing composition of each of the one or more excipients data with the preferred range indicated in the IIG specification. If the one or more excipients composition is determined to exceed the preferred range as per the IIG specification, then the formula generator unit 140 alters the composition of the one or more excipients by recalculating the weight per tablet of the excipients and further continues to compare with the preferred range of the IIG specification to determine the correct set of excipients data that are conforming within the preferred range as per the IIG specification.

The risk assessment unit 150 is configured to determine one or more product challenges anticipated for the pharmaceutical product based on the identified QTPP and CQA data values received from the prediction unit 130 and one or more physiochemical properties that are received from one or more users. The risk assessment unit 150 identifies one or more product challenges and the risk associated with each of the one or more product challenges using the historical database 170. The historical database 170 includes one or more product challenges that are encountered during previous product development, root cause for the one or more product challenges, and one or more CQA's that are affected by the product challenges, wherein the root cause is either due to aberrations in physicochemical properties of the molecule or CPPs and/or CMAs. The risk assessment unit 150 then retrieves a set of parameter values such as CPPs data values and/or CMAs data values and associated values for each of the one or more product challenges identified. The risk assessment unit 150 then retrieves a probability score, a severity score and a detectability score for each of the CPP and CMA values from the historical database 170 and calculates the risk score for each of the one or more product challenges based on the probability score, the severity score, and the detectability score corresponding to each of the CPP data values and CMA data values.

The control unit 160 is configured to identify a functional relationship between the plurality of CMAs data values and CPPs data values and the one or more CQAs data values, identify a range of the values of the plurality of CMAs data and CPPs data that satisfy the identified relationship and desired values of the one or more CQAs by dynamic adjustment of CMAs values and CPPs values. In one embodiment, statistical methods are utilized for the identification of the range of values. In an example, the statistical methods may be one or more of factorial design, response surface design, mixture experiments, Box-Behnken design, Plackett-Burman design, and central composite design methods.

The report generation unit 240 is configured to generate a report based on the identified ranges of CMAs and CPPs values, and also notifies the adjusted data values of CMAs and CPPs and the corresponding risk score to the repository 104 for updating the historical database 170 for developing one or more pharmaceutical products.

In operation, the prediction unit 130 is configured to receive the characteristics of RLDs and the APIs inputted by the one or more users. In one embodiment, the characteristics of RLDs include but not limited to characterization data such as description, batch no., expiry date, strength (mg), average weight (mg), score, coating, diameter (mm), thickness (mm), volume (mm³), hardness (kP), disintegration time (min), disintegration observation, assay (% w/w of label claim), related compound (RC) (%), highest individual unknown, and so on. Upon receiving the characteristics of RLDs, the prediction unit 130 identifies CQAs data values and QTTP data values for the pharmaceutical product to be developed. The prediction unit 130 also predicts the appropriate manufacturing process. In an embodiment, the manufacturing process comprises at least one of wet granulation, dry granulation, direct compression or any other manufacturing process. The prediction unit 130 further determines one or more excipients and composition of the one or more excipients to be used for developing the pharmaceutical product. In an example, for branded Acetriptan 20 mg, the composition determined by the prediction unit 130 is as shown in below table 1:

TABLE 1

| | | Unit (mg | Unit |
|---|---|---|---|
| Component | Function | per tablet) | (% w/w) |
| Acetriptan, USP | Active | 20.0 | 10.0 |
| Lactose Monohydrate, NF | Filler | 64-86 | 32-43 |
| Microcrystalline Cellulose (MCC), | Filler | 72-92 | 36-46 |
| NF | | | |
| Croscarmellose Sodium (CCS), NF | Disintegrant | 2-10 | 1-5 |
| Magnesium Stearate, NF | Lubricant | 2-6 | 1-3 |
| Talc, NF | Glidant/ | 1-10 | 0.5-5 |
| | Lubricant | | |
| Total tablet weight | | 200 | 100 |

The formula generator unit 140 coupled with the prediction unit 130 receives the one or more excipients data associated with the API from the prediction unit 130 and calculates weight per tablet for each of the one or more excipient values. The formula generator unit 140 is further configured to determine qualification of the one or more excipients to be used in the pharmaceutical product development by comparing composition of each of the one or more excipient values with an IIG specification. If the one or more excipients composition is determined to exceed the ranges as per the IIG specification or handbook of excipients limit or any other respective limits that are preconfigured, then the formula generator unit 140 corrects the one or more excipients composition by recalculating the weight per tablet of the corrected excipients and further continues to compare with the IIG specification. For instance, the formula generator 140 determines whether the excipients composition for Acetriptan 20 mg, as shown in the table 1, are within the IIG specification.

Upon obtaining the corrected excipients, the prediction unit 130 may alert the one or more users to develop the pharmaceutical product and receives one or more values associated with properties of the pharmaceutical product thus developed. In an example, the properties can be hardness, friability, weight variation, sticking and other related properties of the pharmaceutical product. The prediction unit 130 receives the properties of the pharmaceutical product as input from the one or more users and determines the acceptance range of the received properties. In one embodiment, the prediction unit 130 determines whether the drug properties are acceptable based on the historical data 170 for a similar pharmaceutical product or RLD. If the drug properties are exceeding the acceptance range, the prediction unit 130 proposes modifications in the manufacturing process steps. In an example, the predicted unit 130 propose to modify the manufacturing process from the direct compression to the wet granulation. If the drug properties are acceptable, the prediction unit 130 alerts the one or more users to subject the pharmaceutical product to drug release process. The observations of the drug release process are inputted to the risk assessment unit 150. The risk assessment unit 150 is configured to determine one or more product challenges anticipated for the pharmaceutical product based on the identified QTPP and CQA data values and one or more physiochemical properties that are received from one or more users. The risk assessment unit 150 determines one or more CPPs and/or CMAs, associated data values for each of the one or more product challenges, and one or more CQA's that are affected by the product challenges.

In an example, the risk assessment unit 150 determines CPPs impacting one or more CQAs such as not limited to impeller speed, tip speed, granulating liquid temperature, wet massing time, pre and post granulation mix time, bowl temperature, powder feed rate, screw speed, binder addition rate, barrel temperature, and so on for different manufacturing processes. The risk assessment unit 150 also determines CMAs impacting one or more CQAs such as particle size, solid form, degree of crystallization, shape and morphology, surface area, moisture content, solubility, contact angle, binder type and grade, diluent type and grade, disintegrant type and grade, granulating fluid viscosity, surface tension, and so on. Upon determining the CPPs and/or CMAs impacting the CQAs, the risk assessment unit 150 determines the risk score for each of the product challenge by multiplying probability (P), severity (S) and detectability (D) scores corresponding to each of the CPP values and CMA values. Upon determining the risk score, the control unit 160 identifies a range of the CPPs values and CMAs values within which desired CQAs can be achieved. The report generation unit 240 then generates a report based on the identified ranges of CMAs and CPPs, and also notifies the adjusted values of CMAs and CPPs and the corresponding risk score to the repository 104 for updating the historical database 170.

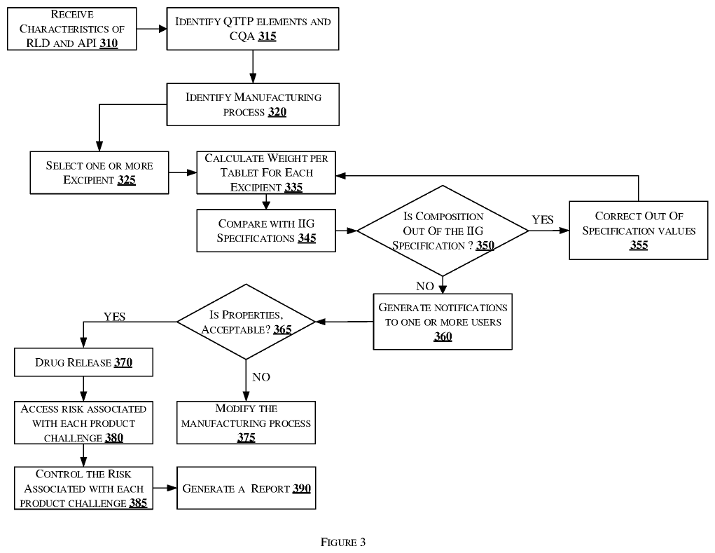

FIG. 3 illustrates an exemplary flowchart for smart product development in accordance with some embodiments of the present disclosure;

As illustrated in FIG. 3, the method 300 comprises one or more blocks implemented by the ROS 102. The method 300 may be described in the general context of computer executable instructions. Generally, computer executable instructions can include routines, programs, objects, components, data structures, procedures, modules, and functions, which perform particular functions or implement particular abstract data types.

The order in which the method 300 is described is not intended to be constructed as a limitation, and any number of the described method blocks can be combined in any order to implement the method 300. Additionally, individual blocks may be deleted from the method 300 without departing from the spirit and scope of the subject matter described herein. Furthermore, the method 300 can be implemented in any suitable hardware, software, firmware, or combination thereof.

The method 300 indicates end to end processes involved in development of the pharmaceutical product with one or more processes being enhanced to optimize resources for the pharmaceutical product development. In one example, FIG. 300 shows the flowchart for development of pharmaceutical product. In the initial step of development at block 310, the prediction unit 130 receives one or more characteristics of RLDs and the APIs inputted by the user. Upon receiving the one or more characteristics of RLDs, one or more CQAs data and QTPP elements data for the pharmaceutical product are identified at block 315. In one embodiment, an appropriate manufacturing process is identified, by verifying if the APIs meet predefined criteria for the pharmaceutical product based on the historical data 170 for the same or similar RLDs at block 320. Once the manufacturing process is determined, one or more suitable excipients data and the composition of the one or more excipients are selected for developing the pharmaceutical product at block 325. The formula generator unit 140 then calculates weight per tablet for each of the one or more excipients data at block 335 and compares the calculated weight with inactive ingredient guide (IIG) specifications to determine qualification of the one or more excipients to be used in the pharmaceuticals product development at block 345. If the formula generator unit 140 determines that the composition of each of the one or more excipients is out of the IIG specification at block 350, then the composition of each of the one or more excipients data that are out of the IIG specification values are corrected at block 355, and the weight of the corrected excipients is recalculated at block 335 and further continues to compare with IIG specifications at block 345 to satisfy the IIG specification values. Upon obtaining the corrected one or more excipient data values, the prediction unit 130 may generate one or more alerts and/or notifications to the one or more users to develop the pharmaceutical product at block 360. The one or more users then develops the pharmaceutical product and determines one or more values associated with properties of the pharmaceutical product. The determined properties of the pharmaceutical product are input to the prediction unit 130 and are compared with acceptance range of one or more values associated with properties of the pharmaceutical product at block 365. If the one or more values associated with the properties exceeds in the acceptable range, then the one or more user is notified to modify the manufacturing process at block 375. If the one or more values associated with the properties are in the acceptable range, then the prediction unit 130 alerts the one or more users to subject the pharmaceutical product to drug release process at block 370. The observations of the drug release process are inputted to the risk assessment unit 150 at block 380 to access risk associated with product challenges by determining one or more product challenges anticipated for the pharmaceutical product based on the identified QTPP and CQA data values and one or more physiochemical properties are received from one or more users using the historical database 170, wherein the historical database 170 includes one or more product challenges that are encountered during previous product development, the root cause identified for the one or more challenges, and one or more CQA's that are affected by the product challenges, and wherein the root cause is either due to physicochemical properties of the molecule or CPPs and/or CMAs. In one embodiment, accessing risk associated with product challenges includes retrieving one or more CPPs and/or CMAs and associated data values for each of the one or more product challenges identified, retrieving a score for each probability, severity and detectability for each of the CPP and CMA values from the historical database and calculating the risk score for each of the one or more product challenges based on the probability score, the severity score, and the detectability score corresponding to each of the CPP data values and CMA data values. The risk score associated with each of the product challenges is provided to the control unit 160 at block 385 to control the risk associated by identifying a functional relationship between the plurality of CMAs data values and CPPs data values and the one or more CQAs data values, and identifying a range of the data values of the plurality of CMAs and CPPs that satisfy the identified relationship and desired data values of the one or more CQAs by dynamical adjustment of CPPs and CMAs. In one embodiment, statistical methods are utilized for the identification of the range of values. In an exemplary embodiment, the statistical methods include factorial design, response surface design, mixture experiments, Box-Behnken design, Plackett-Burman design, central composite design. The report generation unit 240 generates a report based on the identified ranges of CMAs and CPPs data values, and also updates the adjusted data values of CMAs and CPPs and the corresponding risk score in the historical database at block 390. In one embodiment, the ROS 102 also facilitates analytical research and development by developing methods of analysis for the pharmaceutical product. Thus, the one or more R&D processes are enhanced to optimize resources for the pharmaceutical product development with a minimum number of R&D experiments by incorporating a series of steps in a structured manner in all phases of the pharmaceutical product development, thereby reducing unwanted economical burden and also reducing delay in development of the pharmaceutical product. Further, incorporating the series of steps in the structured manner in all phases of the pharmaceutical product development will bring unification, consistency, optimal utilization of resources available in an ordered and timely manner in organization for the development of pharmaceutical product. Also, by maintaining the experimental knowledge and data collected from the plurality of experiments previously conducted in the historical database, reduces fatal human errors thereby enhancing the performance of R&D experimentations.

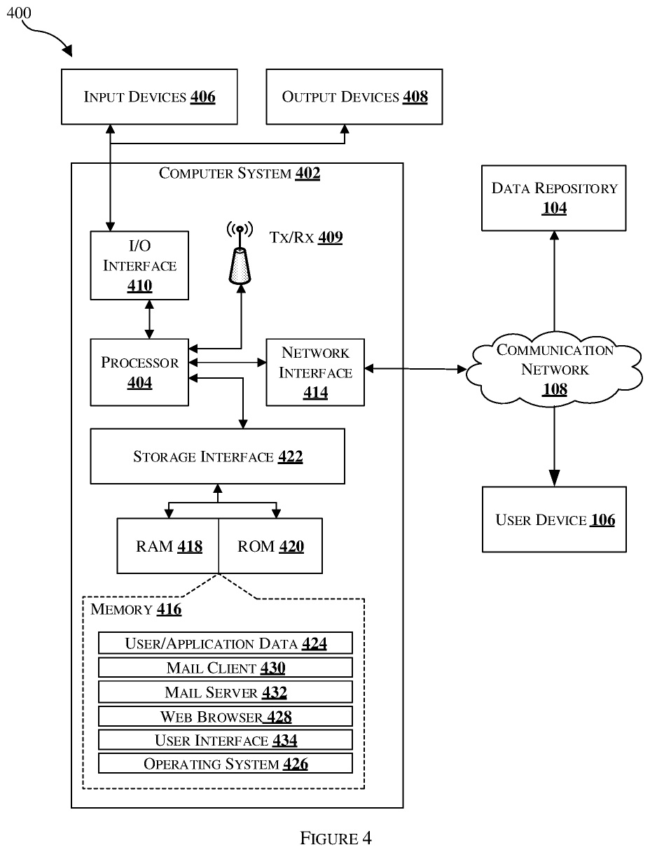

FIG. 4 illustrates a block diagram of an exemplary computer system for implementing embodiments consistent with the present disclosure.

In an embodiment, the computer system 402 may be resource management system (ROS) 102, which is used for enhancing R&D experimentation. The computer system 402 may include a central processing unit (“CPU” or “processor”) 404. The processor 404 may comprise at least one data processor for executing program components for executing user or system-generated business processes. The processor 404 may include specialized processing units such as integrated system (bus) controllers, memory management control units, floating point units, graphics processing units, digital signal processing units, etc.

The processor 404 may be disposed in communication with one or more input/output (I/O) devices (406 and 408) via I/O interface 410. The I/O interface 410 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface 410, the computer system 402 may communicate with one or more I/O devices. For example, the input device 406 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. Output device 408 may be a printer, fax machine, video display (e.g., cathode ray tube (CRT), liquid crystal display (LCD), light-emitting diode (LED), plasma, or the like), audio speaker, etc. In some embodiments, a transceiver 409 may be disposed in connection with the processor 404. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some implementations, the processor 404 may be disposed in communication with a communication network 106 via a network interface 414. The network interface 414 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface 414 and the communication network 108, the computer system 402 may be connected to the data repository 104 and the user device 106.

The communication network 412 can be implemented as one of the several types of networks, such as intranet or any such wireless network interfaces. The communication network 412 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 412 may include a variety of network devices, including routers, bridges, servers, computing devices, storage devices, etc.

In some embodiments, the processor 404 may be disposed in communication with a memory 416 e.g., RAM 418, and ROM 420, etc. as shown in FIG. 4, via a storage interface 422. The storage interface 422 may connect to memory 416 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 416 may store a collection of program or database components, including, without limitation, user/application 424, an operating system 426, a web browser 428, a mail client 430, a mail server 432, a user interface 434, and the like. In some embodiments, computer system 402 may store user/application data 424, such as the data, variables, records, etc. as described in this invention. Such databases may be implemented as fault-tolerant, relational, scalable, secure databases such as Oracle or Sybase.

The operating system 426 may facilitate resource management and operation of the computer system 402. Examples of operating systems include, without limitation, Apple Macintosh™ OS X™, UNIX™, Unix-like system distributions (e.g., Berkeley Software Distribution (BSD), FreeBSD™, Net BSD™, Open BSD™, etc.), Linux distributions (e.g., Red Hat™, Ubuntu™, K-Ubuntu™, etc.), International Business Machines (IBM™) OS/2™, Microsoft Windows™ (XP™, Vista/7/8, etc.), Apple iOS™, Google Android™, Blackberry™ Operating System (OS), or the like. A user interface may facilitate display, execution, interaction, manipulation, or operation of program components through textual or graphical facilities. For example, user interfaces may provide computer interaction interface elements on a display system operatively connected to the computer system 402, such as cursors, icons, check boxes, menus, windows, widgets, etc. Graphical User Interfaces (GUIs) may be employed, including, without limitation, Apple™ Macintosh™ operating systems' Aqua™, IBM™ OS/2™, Microsoft™ Windows™ (e.g., Aero, Metro, etc.), Unix X-Windows™, web interface libraries (e.g., ActiveX, Java, JavaScript, AJAX, HTML, Adobe Flash, etc.), or the like.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

Finally, the language used in the specification has been principally selected for readability and instructional purposes, and it may not have been selected to delineate or circumscribe the inventive subject matter. Accordingly, the disclosure of the embodiments of the disclosure is intended to be illustrative, but not limiting, of the scope of the disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

1. A method of optimizing research and development experimentations, comprising:
receiving, by a resource management system (ROS), characteristics of reference listed drug (RLD) and Active Pharmaceutical Ingredient (API) associated with the RLD;
identifying, by the ROS, a manufacturing process for the pharmaceutical product based on the API and characteristics of RLD received;
generating, by the ROS, notifications to at least one or more users to develop the pharmaceutical product using the API, one or more excipients associated with the API, and the identified manufacturing process;
determining, by the ROS, acceptance range of one or more values associated with properties of the pharmaceutical product; and
optimizing, by the ROS, research and development experimentation based on the determination.

2. The method as claimed in claim 1, wherein the manufacturing process for the pharmaceutical product is identified using a historical database, wherein the historical database includes experimental data collected from a plurality of experiments previously conducted on at least one of the same and similar pharmaceutical products.

3. The method as claimed in claim 1, wherein optimizing research and development experimentations comprising:
receiving the one or more values associated with properties of the pharmaceutical product developed by the one or more users, wherein the properties of the pharmaceutical product include at least one of hardness, friability, weight, and sticking; and
modifying the manufacturing process for the pharmaceutical product upon determination that the one or more values exceed the acceptance range.

4. The method as claimed in claim 1, wherein one or more excipients associated with the API are selected using Inactive Ingredient Guide (IIG) specification, and wherein selecting the one or more excipients comprising:
calculating weight per tablet for each of the one or more excipients; and
verifying the one or more calculated weight per tablet with the IIG specification to determine that the one or more excipients are acceptable for the pharmaceutical product development.

5. The method as claimed in claim 2, further comprising:
identifying at least one of quality target product profile (QTPP) and critical quality attributes (CQA) data values for the pharmaceutical product based on the received characteristics of RLD;
determining one or more product challenges anticipated for the pharmaceutical product based on the identified QTTP and CQA data values, and one or more physiochemical properties received from one or more user; and
computing a risk score for each of the one or more product challenges based on historic risk score information stored in the historical database.

6. The method as claimed in claim 5, wherein computing the risk score for each of the one or more product challenges comprising:
retrieving a set of parameter values associated with each of the one or more product challenges, wherein the set of parameter values includes at least one of CPP and CMA data values;
retrieving a score for each probability, severity and detectability for each of the set of parameter values based on the historic score information; and
calculating the risk score for each of the one or more product challenges based on the probability score, the severity score, and the detectability score corresponding to each of the set of parameters.

7. The method as claimed in claim 6, further comprising:
identifying a functional relationship between a plurality of CMAs values and CPPs values and the one or more CQAs values;
dynamically adjusting the values of the plurality of CMAs and CPPs to satisfy the identified functional relationship and desired values of the one or more CQAs; and
generating a report based on the adjusted values of CMAs and CPPs that satisfy the desired values of the one or more CQAs; and
updating the adjusted values of CMAs and CPPs and the corresponding risk score in the historical database.

8. A system to optimize research and development experimentations, comprising:
a memory; and
a processor, coupled to the memory, and is configured to:
receive characteristics of reference listed drug (RLD) and Active Pharmaceutical Ingredient (API) associated with the RLD;
identify a manufacturing process for the pharmaceutical product based on the API and characteristics of RLD received;
generate notifications to at least one or more users to develop the pharmaceutical product using the API, one or more excipients associated with the API, and the identified manufacturing process;
determine acceptance range of one or more values associated with properties of the pharmaceutical product; and
optimize research and development experimentations based on the determination.

9. The system as claimed in claim 8, wherein the manufacturing process for the pharmaceutical product is identified using a historical database, wherein the historical database includes experimental data collected from a plurality of experiments previously conducted on at least one of the same and similar pharmaceutical products.

10. The system as claimed in claim 8, wherein to optimize research and development experimentations, the processor is configured to:
receive the one or more values associated with properties of the pharmaceutical product developed by the one or more users, wherein the properties of the pharmaceutical product comprise at least one of hardness, friability, weight, and sticking; and
modify the manufacturing process for the pharmaceutical product upon determination that the one or more values exceed the acceptance range.

11. The system as claimed in claim 8, wherein the one or more excipients associated with the API are selected using Inactive Ingredient Guide (IIG) specification, and wherein to select one or more excipients, the processor is configured to:
calculate weight per tablet for each of the one or more excipients; and
verify the one or more calculated weight per tablet with the IIG specification to determine that the one or more excipients are acceptable for the pharmaceutical product development.

12. The system as claimed in claim 9, wherein the processor is further configured to:
identify at least one of quality target product profile (QTPP) and critical quality attributes (CQA) values for the pharmaceutical product based on the received characteristics of RLD;
determine one or more product challenges anticipated for the pharmaceutical product based on the identified QTTP and CQA values, and one or more physiochemical properties received from the one or more user; and
compute a risk score for each of the one or more product challenges based on historic risk score information stored in the historical database.

13. The system as claimed in claim 12, wherein to computing the risk score for each of the one or more product challenges, the processor is configured to:
retrieve a set of parameter values associated with each of the one or more product challenges, wherein the set of parameter values includes at least one of CPP and CMA values;
retrieve for each of the set of parameter values a score for each probability, severity and detectability based on the historic risk score information; and
calculate the risk score for each of the one or more product challenges based on the probability score, the severity score, and the detectability score corresponding to each of the set of parameters.

14. The system as claimed in claim 13, the processor is further configured to:
identify a functional relationship between a plurality of CMAs values and CPPs values and the one or more CQAs values;
dynamically adjust the values of the plurality of CMAs and CPPs to satisfy the identified functional relationship and desired values of the one or more CQAs; and
generate a report based on the adjusted values of CMAs and CPPs that satisfy the desired values of the one or more CQAs; and
update the adjusted values of CMAs and CPPs and the corresponding risk score in the historical database.

15. The system as claimed in claim 8, wherein the processor is coupled to at least one of a prediction unit, a formula generator unit, a risk assessment unit, and a strategy unit.
